(19) United States
(12) Patent Application Publication
(10) Pub. No.: US 2025/0154099 A1
(43) Pub. Date: May 15, 2025

(54) PREPARATION METHOD OF 2-PHENYLINDOLE DERIVATIVES AND APPLICATION THEREOF

(71) Applicant: Shihezi University, Shihezi (CN)

(72) Inventors: Xinhui Pan, Shihezi (CN); Ke Yang, Shihezi (CN); Ming Zhang, Jinan (CN); Wei Wei, Shihezi (CN); Qiaohui Liu, Shihezi (CN); Ke Zhang, Shihezi (CN); Caiyun Yang, Shihezi (CN); Guanzhong Chen, Shihezi (CN)

(21) Appl. No.: 18/261,340

(22) PCT Filed: Feb. 23, 2023
(86) PCT No.: PCT/CN2023/077760

§ 371 Date: Jul. 13, 2023

(30) Foreign Application Priority Data
Aug. 3, 2022 (CN) 202210927289.6

(51) Int. Cl. C07D 209/08; A61K 31/404; A61K 31/4196; C07D 209/10; C07D 209/12; C07D 405/04

(52) U.S. Cl. CPC C07D 209/08; A61K 31/404; A61K 31/4196; C07D 209/10; C07D 209/12; C07D 405/04

(57) ABSTRACT

The present invention discloses a preparation method and application of 2-phenylindole derivatives. Palladium acetate and copper acetate are added into a glacial acetic acid solution of an indole derivative (formula I) and an arylphenylboronic acid derivative (formula II), stirred at room temperature, and purified to obtain a 2-phenylindole derivative (formula III). The present invention provides a method for direct one-step synthesis of 2-arylindole compounds from indole derivatives and arylphenylboronic acid derivatives as raw materials. The present invention replaces oxygen with air to participate in an oxidation system, expands substituents of indole ring R¹, adds substitution of 5-bit, 6-bit and 7-bit electron withdrawing groups and electron donating groups, further expands the range of substrates, and increases the yield of substitution on indole ring R1 by about 13%, wherein 2-methyl and 3-methoxy substituted by R² and 6-chlorine substituted by R¹ have good antifungal activity.

### TECHNICAL FIELD

The present invention relates to the field of medicine, and in particular to a preparation method of 2-phenylindole derivatives and application thereof.

### BACKGROUND

The development of drugs against drug-resistant fungi has been an ongoing challenge in today's society. At present, available drugs for treating fungal infection comprise amphotericin B, a macrolide polyene: flucytosine that interacts with fungal membrane sterol, another fluoropyrimidine that interacts with mycoprotein and DNA biosynthesis, and multiple azole antifungal drugs (e.g. ketoconazole, itraconazole and fluconazole) that inhibit the biosynthesis of fungal membrane-sterol. The development of antifungal drugs is slower than that of antibacterial drugs, mainly because many fungi have certain characteristics of eukaryotic cells, which makes it difficult to select drugs. Most of these fungi have become resistant to first-line antifungal agents such as azoles and polyenes, thereby hindering proper treatment and/or prevention of the diseases. With the continuous increase of incidence and mortality of fungal infection in immunocompromised patients, it is extremely urgent to find a sensitive, broad-spectrum and safe antifungal drug. In recent years, with the development of molecular mycology, a series of antifungal compounds with novel mechanisms of action have been discovered.

Indole alkaloids have wide biological activities, such as anti-bacteria, anti-virus, anti-tumor and anti-anxiety. In recent years, the antifungal activity of indole derivatives has gradually attracted attention, and the indole derivatives are used for developing novel antifungal compounds. The molecular structures of well-known drugs such as sumatriptan, tadalafil, fluvastatin and rizatriptan are based on indole. An essential amino acid-tryptophan in animals is a derivative of indole. Some natural substances with strong physiological activity, such as alkaloids and auxins, are derivatives of indole. Indole schiff base derivatives are used to resist HIV-1 (Wenqiang Tang et al., Fine Chemicals: p. 1-10.); and 2-phenylindole compounds also have wide biological activities, such as anti-Mycobacterium tuberculosis activity. With the continuous deepening of research on such compounds, there will be more antifungal drugs of indole derivatives for clinical use.

At present, the traditional method of arylation of C—H bonds at C2 site of indole is to halogenate C—H bonds of indole through catalysis by transition metal and then to conduct a cross-coupling reaction to obtain an arylindole compound. The arylation method of C—H bonds at C2 site of indole catalyzed by Pd is reported successively. However, due to the existence of halide, the method increases the reaction cost, and is environmentally unfriendly and inconsistent with the development requirements of green chemistry. In 2008, Shi research group reported a new method for synthesis of 2-phenylindole compounds. This method uses oxygen as an oxidant, and catalyzes the cross-coupling of the indole derivatives and various arylboronic acids by Pd to directly build diaryl C—C bonds. The reaction can be conducted efficiently only at room temperature. Although Shi has fully researched the substitution of benzene ring R1, the research on indole ring R2 is mainly 5-site substitution, and the range of substrates is narrow. In 2017, Jiang research group reported a direct C2 arylation method of free (N—H) indole catalyzed by Pd based on norbornene mediated regioselective C—H activation. The method has high regioselectivity, but the yield of electron withdrawing group substituted products of the indole ring is low, only 26%-50%, and the range of substrates is narrow.

### SUMMARY

In view of this, the present invention provides a method for direct one-step synthesis of 2-arylindole compounds from indole and arylphenylboronic acid as raw materials with a purpose of developing green and efficient synthesis methods with wide applicability of substrates. Compared with the method of Shi research group, this method replaces oxygen with air to participate in an oxidation system. It should be noted that this method expands substituents of indole ring R2, adds substitution of 5-bit, 6-bit and 7-bit electron withdrawing groups and electron donating groups, directly synthesizes various 2-arylindole compounds by one step, further expands the range of substrates, increases the yield of electron withdrawing group substitution on benzene ring R2 from 53% to 87%, and increases the yield of substitution on indole ring R1 by about 13%, wherein 2-methyl and 3-methoxy substituted by R2 and 6-chlorine substituted by R1 have good antifungal activity.

To achieve the above purpose, the present invention adopts the following technical solution:

A preparation method of 2-phenylindole derivatives comprises: adding palladium acetate and copper acetate into a glacial acetic acid solution of formula I and formula II, stirring at room temperature, and purifying to obtain a target product (formula III):

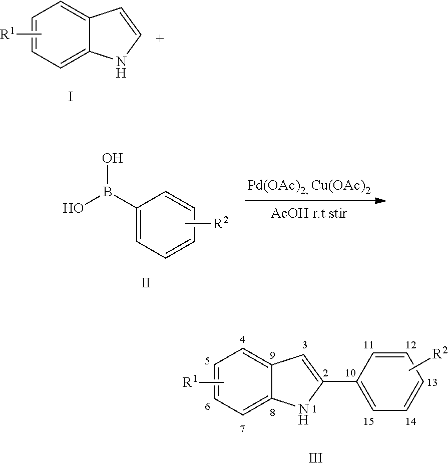

wherein R¹ is substituted at 5, 6 or 7 site, and substituents are selected from F, Cl, OCH₃, CH₃, COOCH₃ and N—CH₃;
R² is substituted at 11, 12, 13 or 14 site, and substituents are selected from F, Cl, CH₃, OCH₃, CF₃, NO₂ or CH₂O₂.

Preferably, in the above preparation method, a molar ratio of the formula I: formula II in the glacial acetic acid solution of the formula I and the formula II is 1: 1-10.

Preferably, in the above preparation method, a molar ratio of the formula I: palladium acetate is 1: 0.1-0.8; and a molar ratio of the formula I: copper acetate is 1: 0.1-0.8.

Preferably, in the above preparation method, stirring time is 6-10 h.

Preferably, in the above preparation method, the purification specifically comprises: conducting rotary evaporation for the reaction liquid after the end of the reaction for rotary evaporation of redundant glacial acetic acid and recovering, adding dichloromethane into obtained black solid for diluting, then extracting the mixture with saturated sodium bicarbonate solution, drying the organic extract with anhydrous magnesium sulfate, filtering, conducting rotary evaporation and concentration, and finally separating and purifying by column chromatography silica gel to obtain a target product.

Preferably, in the above preparation method, the separation of the column chromatography silica gel uses silica gel of 200-300 meshes as a stationary phase, and a mixed solvent of ethyl acetate and petroleum ether in different proportions is used as eluent.

The present invention further discloses an application of the 2-phenylindole derivative prepared by the above preparation method in preparation of antifungal reagents or antibacterial reagents.

Preferably, in the above application, fungi comprise Candida albicans, Cryptococcosis and Aspergillus fumigatus Fresenius; and bacteria comprise Staphylococcus aureus, Escherichia coli or Pseudomonas aeruginosa Migula.

Preferably, in the above application, the fungi are drug-resistant bacteria or sensitive bacteria; the antifungal reagents also comprise azole antifungal drugs; the azole antifungal drugs comprise ketoconazole, itraconazole or fluconazole; and a mass ratio of the azole antifungal drugs to the 2-phenylindole derivative is 1-16: 64-4.

Compared with the prior art, the present invention has the beneficial effects that:

The present invention conducts optimization based on Suzuki coupling reaction, so that Pd(II) catalyzes indole C (sp²)-H and phenylboronic acid aryl for direct C(sp²)-(sp²) coupling without dependence on C-X activation, which has the advantage that the C—C can be directly coupled to produce 2-phenylindole without using indole halide, thereby lowering the cost and reducing environmental pollution. The mechanism diagram of the reaction of the present invention is as follows:

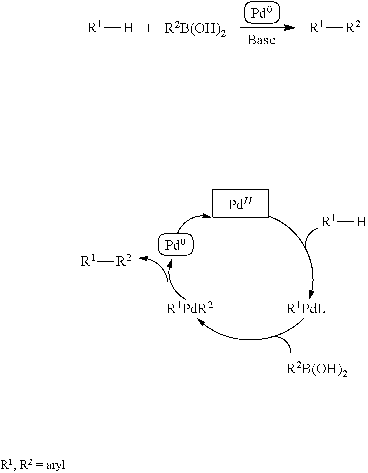

The present invention is applicable to various different electron withdrawing groups and electron donating groups, simple in operation and mild in conditions.

Compared with the traditional synthesis method for 2-phenylindole, the present invention improves the catalyst, adds copper acetate to form a palladium-copper bimetallic composite catalyst, obviously enhances the catalysis effect, can reduce reaction conditions from oxygen to air conditions, and keeps a high yield.

The combination of the compound of the present invention and the azole drugs shows a synergistic effect on inhibiting the proliferation of clinical multi-drug resistant bacteria CA10 or can improve the sensitivity of the clinical multi-drug resistant bacteria to the azole drugs, so that the combination concentration is reduced to be less than the drug-resistant concentration, and the effect of reversing the drug resistance can be achieved.

### DESCRIPTION OF DRAWINGS

To more clearly describe the technical solution in the embodiments of the present invention or in the prior art, the drawings required to be used in the description of the embodiments or the prior art will be simply presented below. Apparently, the drawings in the following description are merely the embodiments of the present invention, and for those ordinary skilled in the art, other drawings can also be obtained according to the provided drawings without contributing creative labor.

FIG. 1 shows the influence of compound 2 on RAW 264.7 cell viability;

FIG. 2 shows the influence of compound 4 on RAW 264.7 cell viability;

FIG. 3 shows the influence of compound 5 on RAW 264.7 cell viability.

### DETAILED DESCRIPTION

The technical solutions in embodiments of the present invention will be clearly and fully described below. Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

### Embodiment 1

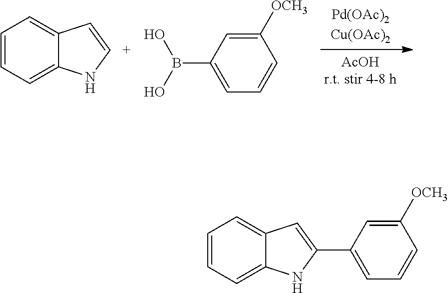 [SMILES: c1ccc2[nH]ccc2c1 ; COc1cccc(B(O)O)c1 ; COc1cccc(-c2cc3ccccc3[nH]2)c1]

Catalytic synthesis method: adding palladium acetate (0.5 equiv.) and copper acetate (0.5 equiv.) into 20 mL of glacial acetic acid solution of indole (1 equiv.) and 3-methoxyphenylboronic acid (2 equiv.) in an air atmosphere, stirring at room temperature to react for 8 h, and detecting the reaction by TLC until the reaction is completed; conducting rotary evaporation for the reaction liquid for rotary evaporation of redundant glacial acetic acid and recovery; adding dichloromethane into the obtained black solid for dilution, and then extracting the mixture with (20 mL×3) saturated sodium bicarbonate solution; drying the organic extract with anhydrous magnesium sulfate, filtering, conducting rotary evaporation and concentration, separating by column chromatography, purifying and separating the product by taking silica gel of 200-300 meshes as a stationary phase and a mixed solvent of ethyl acetate and petroleum ether in different proportions as eluent, concentrating and drying to obtain white powder. Physical properties and representation data of the obtained compound are as follows. The physical properties and the representation data of the obtained compound are as follows: yield 51%, m.p. 139.4-140.7° C.; ¹H NMR (400 MHz, CDCl₃) δ 8.30 (s, 1H), 7.62 (d, J=7.7 Hz, 1H), 7.39-7.29 (m, 2H), 7.23-7.09 (m, 4H), 6.94-6.73 (m, 2H), 3.85 (s, 3H). HRMS (EI) m/z calcd for C₁₅H₁₄NO [M+H]⁺ 224.1075, found 224.1070.

### Embodiment 2

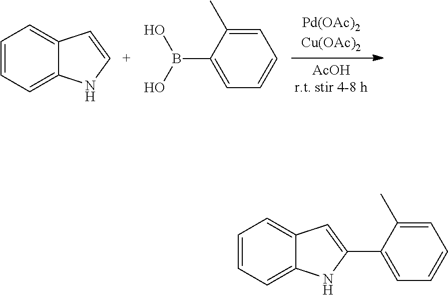

Catalytic synthesis method: adding palladium acetate (0.5 equiv.) and copper acetate (0.5 equiv.) into 20 mL of glacial acetic acid solution of indole (1 equiv.) and 2-methylbenzeneboronic acid (10 equiv.) in an air atmosphere, stirring at room temperature to react for 8 h, and detecting the reaction by TLC until the reaction is completed; conducting rotary evaporation for the reaction liquid for rotary evaporation of redundant glacial acetic acid and recovery; adding dichloromethane into the obtained black solid for dilution, and then extracting the mixture with (20 mL×3) saturated sodium bicarbonate solution; drying the organic extract with anhydrous magnesium sulfate, filtering, conducting rotary evaporation and concentration, separating by column chromatography, purifying and separating the product by taking silica gel of 200-300 meshes as a stationary phase and a mixed solvent of ethyl acetate and petroleum ether in different proportions as eluent, concentrating and drying to obtain white powder. Physical properties and representation data of the obtained compound are as follows. The physical properties and the representation data of the obtained compound are as follows: yield 49.3%, m.p. 93.0-94.3° C.; ¹H NMR (400 MHz, CDCl₃) δ 8.05 (s, 1H), 7.58 (d, J=7.8 Hz, 1H), 7.40 (dd, J=5.1, 3.8 Hz, 1H), 7.33 (d, J=7.2 Hz, 1H), 7.23-7.17 (m, 3H), 7.16-7.11 (m, 1H), 7.09-7.04 (m, 1H), 6.54 (dd, J=2.1, 0.8 Hz, 1H), 2.43 (s, 3H). HRMS (EI) m/z calcd for C₁₅H₁₃N [M+H]⁺ 208.1126, found 208.1129.

### Embodiment 3

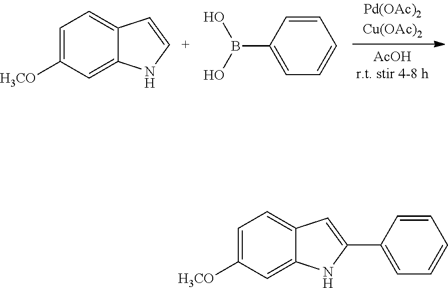 [SMILES: COc1ccc2cc[nH]c2c1 ; OB(O)c1ccccc1 ; COc1ccc2cc(-c3ccccc3)[nH]c2c1]

Catalytic synthesis method: adding palladium acetate (0.5 equiv.) and copper acetate (0.5 equiv.) into 20 mL of glacial acetic acid solution of 6-methoxyindole (1 equiv.) and phenylboronic acid (2 equiv.) in an air atmosphere, stirring at room temperature to react for 8 h, and detecting the reaction by TLC until the reaction is completed; conducting rotary evaporation for the reaction liquid for rotary evaporation of redundant glacial acetic acid and recovery; adding dichloromethane into the obtained black solid for dilution, and then extracting the mixture with (20 mL×3) saturated sodium bicarbonate solution; drying the organic extract with anhydrous magnesium sulfate, filtering, conducting rotary evaporation and concentration, separating by column chromatography, purifying and separating the product by taking silica gel of 200-300 meshes as a stationary phase and a mixed solvent of ethyl acetate and petroleum ether in different proportions as eluent, concentrating and drying to obtain white powder. Physical properties and representation data of the obtained compound are as follows. The physical properties and the representation data of the obtained compound are as follows: yield 87%, m.p. 173.0-176.0° C.; ¹H NMR (400 MHz, DMSO) δ 11.35 (s, 1H), 7.85-7.75 (m, 2H), 7.42 (dd, J=14.8, 8.0 Hz, 3H), 7.28 (d, J=7.4 Hz, 1H), 6.89 (d, J=2.1 Hz, 1H), 6.81 (d, J=1.8 Hz, 1H), 6.67 (dd, J=8.6, 2.3 Hz, 1H), 3.79 (s, 3H). HRMS (EI) m/z calcd for C₁₅H₁₄NO [M+H]⁺ 224.1075, found 224.1087.

### Embodiment 4

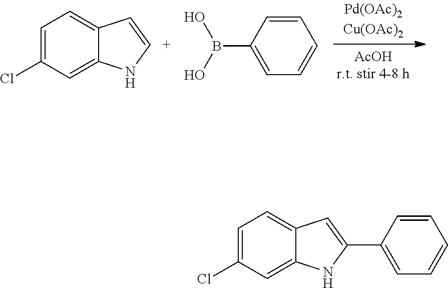 [SMILES: Clc1ccc2cc[nH]c2c1 ; OB(O)c1ccccc1 ; Clc1ccc2cc(-c3ccccc3)[nH]c2c1]

Catalytic synthesis method: adding palladium acetate (0.5 equiv.) and copper acetate (0.5 equiv.) into 20 mL of glacial acetic acid solution of 6-chloroindole (1 equiv.) and phenylboronic acid (2 equiv.) in an air atmosphere, stirring at room temperature to react for 8 h, and detecting the reaction by TLC until the reaction is completed; conducting rotary evaporation for the reaction liquid for rotary evaporation of redundant glacial acetic acid and recovery; adding dichloromethane into the obtained black solid for dilution, and then extracting the mixture with (20 mL×3) saturated sodium bicarbonate solution; drying the organic extract with anhydrous magnesium sulfate, filtering, conducting rotary evaporation and concentration, separating by column chromatography, purifying and separating the product by taking silica gel of 200-300 meshes as a stationary phase and a mixed solvent of ethyl acetate and petroleum ether in different proportions as eluent, concentrating and drying to obtain white powder. Physical properties and representation data of the obtained compound are as follows. The physical properties and the representation data of the obtained compound are as follows: yield 63%, m.p. 182.0-183.0° C.; ¹H NMR (400 MHz, DMSO) δ 11.70 (s, 1H), 7.88-7.82 (m, 2H), 7.54 (d, J=8.4 Hz, 1H), 7.47 (t, J=7.7 Hz, 2H), 7.40 (d, J=1.7 Hz, 1H), 7.33 (t, J=7.4 Hz, 1H), 7.01 (dd, J=8.4, 1.9 Hz, 1H), 6.93 (d, J=1.5 Hz, 1H). HRMS (EI) m/z calcd for C₁₄H₁₁ClN [M+H]⁺ 228.0508, found 228.0512.

### Embodiment 5

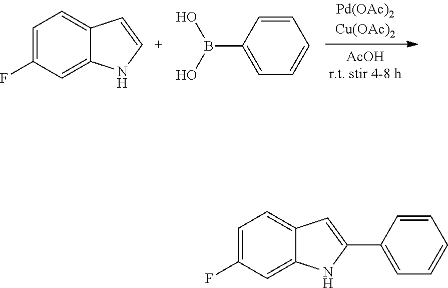 [SMILES: Fc1ccc2cc[nH]c2c1 ; OB(O)c1ccccc1 ; Fc1ccc2cc(-c3ccccc3)[nH]c2c1]

Catalytic synthesis method: adding palladium acetate (0.5 equiv.) and copper acetate (0.5 equiv.) into 20 mL of glacial acetic acid solution of 6-fluoroindole (1 equiv.) and phenylboronic acid (2 equiv.) in an air atmosphere, stirring at room temperature to react for 8 h, and detecting the reaction by TLC until the reaction is completed; conducting rotary evaporation for the reaction liquid for rotary evaporation of redundant glacial acetic acid and recovery; adding dichloromethane into the obtained black solid for dilution, and then extracting the mixture with (20 mL×3) saturated sodium bicarbonate solution; drying the organic extract with anhydrous magnesium sulfate, filtering, conducting rotary evaporation and concentration, separating by column chromatography, purifying and separating the product by taking silica gel of 200-300 meshes as a stationary phase and a mixed solvent of ethyl acetate and petroleum ether in different proportions as eluent, concentrating and drying to obtain white powder. Physical properties and representation data of the obtained compound are as follows. The physical properties and the representation data of the obtained compound are as follows: yield 65%, m.p. 171.0-172.0° C.; ¹H NMR (400 MHz, CDCl₃) δ 8.35 (s, 1H), 7.64 (d, J=7.9 Hz, 2H), 7.56-7.42 (m, 3H), 7.35-7.31 (m, 1H), 7.09 (d, J=9.4 Hz, 1H), 6.93-6.85 (m, 1H), 6.80 (s, 1H). HRMS (EI) m/z calcd for C₁₄H₁₁FN [M+H]⁺ 212.0876, found 212.0872.0.

### Embodiment 6

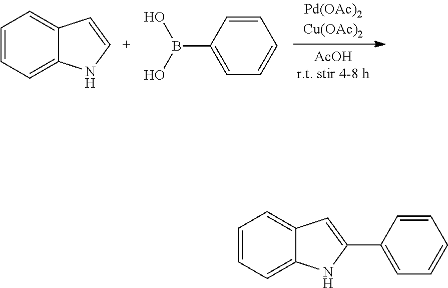 [SMILES: c1ccc2[nH]ccc2c1 ; OB(O)c1ccccc1 ; c1ccc(-c2cc3ccccc3[nH]2)cc1]

Catalytic synthesis method: adding palladium acetate (0.5 equiv.) and copper acetate (0.5 equiv.) into 20 mL of glacial acetic acid solution of indole (1 equiv.) and phenylboronic acid (1 equiv.) in an air atmosphere, stirring at room temperature to react for 8 h, and detecting the reaction by TLC until the reaction is completed; conducting rotary evaporation for the reaction liquid for rotary evaporation of redundant glacial acetic acid and recovery; adding dichloromethane into the obtained black solid for dilution, and then extracting the mixture with (20 mL×3) saturated sodium bicarbonate solution; drying the organic extract with anhydrous magnesium sulfate, filtering, conducting rotary evaporation and concentration, separating by column chromatography, purifying and separating the product by taking silica gel of 200-300 meshes as a stationary phase and a mixed solvent of ethyl acetate and petroleum ether in different proportions as eluent, concentrating and drying to obtain white powder. Physical properties and representation data of the obtained compound are as follows. The physical properties and the representation data of the obtained compound are as follows: yield 84%, m.p. 193.4-193.9° C.; ¹H NMR (400 MHz, DMSO) δ 11.52 (s, 1H), 7.85 (dd, J=8.3, 1.1 Hz, 2H), 7.56-7.37 (m, 4H), 7.32 (d, J=7.4 Hz, 1H), 7.14-7.05 (m, 1H), 7.00 (dd, J=7.9, 0.9 Hz, 1H), 6.89 (dd, J=2.1, 0.7 Hz, 1H). HRMS (EI) m/z calcd for C₁₄H₁₂N [M+H]⁺ 194.0970, found 194.0973.

### Embodiment 7

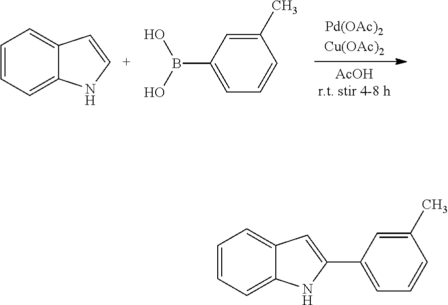

Catalytic synthesis method: adding palladium acetate (0.5 equiv.) and copper acetate (0.5 equiv.) into 20 mL of glacial acetic acid solution of indole (1 equiv.) and 3-methylbenzeneboronic acid (2 equiv.) in an air atmosphere, stirring at room temperature to react for 8 h, and detecting the reaction by TLC until the reaction is completed; conducting rotary evaporation for the reaction liquid for rotary evaporation of redundant glacial acetic acid and recovery; adding dichloromethane into the obtained black solid for dilution, and then extracting the mixture with (20 mL×3) saturated sodium bicarbonate solution; drying the organic extract with anhydrous magnesium sulfate, filtering, conducting rotary evaporation and concentration, separating by column chromatography, purifying and separating the product by taking silica gel of 200-300 meshes as a stationary phase and a mixed solvent of ethyl acetate and petroleum ether in different proportions as eluent, concentrating and drying to obtain white powder. Physical properties and representation data of the obtained compound are as follows. The physical properties and the representation data of the obtained compound are as follows: yield 45%, m.p. 138.5-139° C.; ¹H NMR (400 MHz, Acetone) δ 10.62 (s, 1H), 7.73-7.62 (m, 2H), 7.60-7.52 (m, 1H), 7.44-7.38 (m, 1H), 7.33 (t, J=7.7 Hz, 1H), 7.16-7.06 (m, 2H), 7.02 (ddd, J=8.0, 7.1, 1.1 Hz, 1H), 6.88 (dd, J=2.2, 0.8 Hz, 1H), 2.39 (s, 3H). HRMS (EI) m/z calcd for C₁₅H₁₃N [M+H]⁺ 208.1118, found 208.1126.

### Embodiment 8

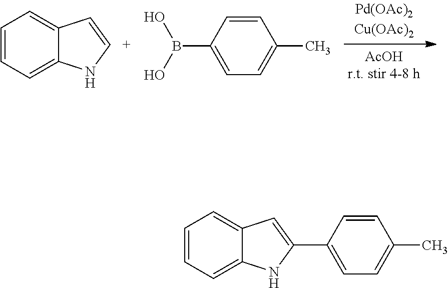

Catalytic synthesis method: adding palladium acetate (0.5 equiv.) and copper acetate (0.5 equiv.) into 20 mL of glacial acetic acid solution of indole (1 equiv.) and 4-methylbenzeneboronic acid (1 equiv.) in an air atmosphere, stirring at room temperature to react for 8 h, and detecting the reaction by TLC until the reaction is completed; conducting rotary evaporation for the reaction liquid for rotary evaporation of redundant glacial acetic acid and recovery; adding dichloromethane into the obtained black solid for dilution, and then extracting the mixture with (20 mL×3) saturated sodium bicarbonate solution; drying the organic extract with anhydrous magnesium sulfate, filtering, conducting rotary evaporation and concentration, separating by column chromatography, purifying and separating the product by taking silica gel of 200-300 meshes as a stationary phase and a mixed solvent of ethyl acetate and petroleum ether in different proportions as eluent, concentrating and drying to obtain white powder. Physical properties and representation data of the obtained compound are as follows. The physical properties and the representation data of the obtained compound are as follows: yield 56%, m.p. 220.0-221.0° C.; ¹H NMR (400 MHz, Acetone) δ 10.59 (s, 1H), 7.79-7.70 (m, 2H), 7.55 (d, J=8.2 Hz, 1H), 7.40 (dd, J=8.1, 0.8 Hz, 1H), 7.26 (d, J=7.9 Hz, 2H), 7.09 (ddd, J=8.2, 7.1, 1.2 Hz, 1H), 7.01 (ddd, J=8.0, 7.1, 1.1 Hz, 1H), 6.84 (dd, J=2.2, 0.8 Hz, 1H), 2.35 (s, 3H). HRMS (EI) m/z calcd for C₁₅H₁₃N [M+H]⁺ 208.1136, found 208.1126.

### Embodiment 9

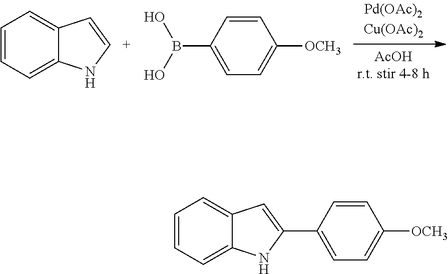 [SMILES: c1ccc2[nH]ccc2c1 ; COc1ccc(B(O)O)cc1 ; COc1ccc(-c2cc3ccccc3[nH]2)cc1]

Catalytic synthesis method: adding palladium acetate (0.5 equiv.) and copper acetate (0.5 equiv.) into 20 mL of glacial acetic acid solution of indole (1 equiv.) and 4-methoxyphenylboronic acid (1 equiv.) in an air atmosphere, stirring at room temperature to react for 8 h, and detecting the reaction by TLC until the reaction is completed; conducting rotary evaporation for the reaction liquid for rotary evaporation of redundant glacial acetic acid and recovery; adding dichloromethane into the obtained black solid for dilution, and then extracting the mixture with (20 mL×3) saturated sodium bicarbonate solution; drying the organic extract with anhydrous magnesium sulfate, filtering, conducting rotary evaporation and concentration, separating by column chromatography, purifying and separating the product by taking silica gel of 200-300 meshes as a stationary phase and a mixed solvent of ethyl acetate and petroleum ether in different proportions as eluent, concentrating and drying to obtain white powder. Physical properties and representation data of the obtained compound are as follows. The physical properties and the representation data of the obtained compound are as follows: yield 40%, m.p. 232.0-233.0° C.; ¹H NMR (400 MHz, Acetone) δ 10.53 (s, 1H), 7.89-7.70 (m, 2H), 7.53 (d, J=7.8 Hz, 1H), 7.38 (dd, J=8.0, 0.8 Hz, 1H), 7.10-6.97 (m, 4H), 6.76 (dd, J=2.2, 0.8 Hz, 1H), 3.84 (s, 3H). HRMS (EI) m/z calcd for C₁₅H₁₄NO [M+H]⁺ 224.1070, found 224.1075.

### Embodiment 10

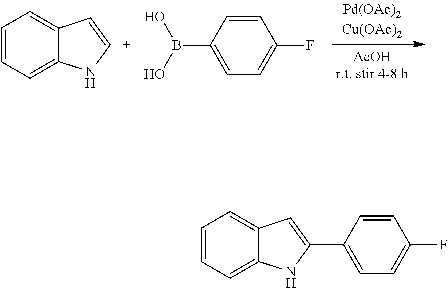 [SMILES: c1ccc2[nH]ccc2c1 ; OB(O)c1ccc(F)cc1 ; Fc1ccc(-c2cc3ccccc3[nH]2)cc1]

Catalytic synthesis method: adding palladium acetate (0.5 equiv.) and copper acetate (0.5 equiv.) into 20 mL of glacial acetic acid solution of indole (1 equiv.) and 4-flurorphenylboronic acid (1 equiv.) in an air atmosphere, stirring at room temperature to react for 8 h, and detecting the reaction by TLC until the reaction is completed; conducting rotary evaporation for the reaction liquid for rotary evaporation of redundant glacial acetic acid and recovery; adding dichloromethane into the obtained black solid for dilution, and then extracting the mixture with (20 mL×3) saturated sodium bicarbonate solution; drying the organic extract with anhydrous magnesium sulfate, filtering, conducting rotary evaporation and concentration, separating by column chromatography, purifying and separating the product by taking silica gel of 200-300 meshes as a stationary phase and a mixed solvent of ethyl acetate and petroleum ether in different proportions as eluent, concentrating and drying to obtain white powder. Physical properties and representation data of the obtained compound are as follows. The physical properties and the representation data of the obtained compound are as follows: yield 73%, m.p. 191.0-193.0° C.; ¹H NMR (400 MHz, Acetone) δ 10.64 (s, 1H), 7.92-7.85 (m, 2H), 7.56 (d, J=7.9 Hz, 1H), 7.41 (dd, J=8.1, 0.8 Hz, 1H), 7.26-7.19 (m, 2H), 7.11 (ddd, J=8.2, 7.1, 1.2 Hz, 1H), 7.03 (ddd, J=8.0, 7.1, 1.0 Hz, 1H), 6.86 (dd, J=2.1, 0.7 Hz, 1H). HRMS (EI) m/z calcd for C₁₄H₁₁FN [M+H]⁺ 212.0876, found 212.0876.

### Embodiment 11

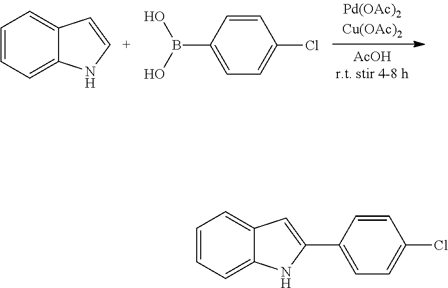 [SMILES: c1ccc2[nH]ccc2c1 ; OB(O)c1ccc(Cl)cc1 ; Clc1ccc(-c2cc3ccccc3[nH]2)cc1]

Catalytic synthesis method: adding palladium acetate (0.3 equiv.) and copper acetate (0.5 equiv.) into 20 mL of glacial acetic acid solution of indole (1 equiv.) and 4-chlorophenylboronic acid (1 equiv.) in an air atmosphere, stirring at room temperature to react for 8 h, and detecting the reaction by TLC until the reaction is completed; conducting rotary evaporation for the reaction liquid for rotary evaporation of redundant glacial acetic acid and recovery; adding dichloromethane into the obtained black solid for dilution, and then extracting the mixture with (20 mL×3) saturated sodium bicarbonate solution; drying the organic extract with anhydrous magnesium sulfate, filtering, conducting rotary evaporation and concentration, separating by column chromatography, purifying and separating the product by taking silica gel of 200-300 meshes as a stationary phase and a mixed solvent of ethyl acetate and petroleum ether in different proportions as eluent, concentrating and drying to obtain white powder. Physical properties and representation data of the obtained compound are as follows. The physical properties and the representation data of the obtained compound are as follows: yield 79%, m.p. 196.0-197.0° C.; ¹H NMR (400 MHz, Acetone) δ 10.70 (s, 1H), 7.90-7.84 (m, 2H), 7.57 (d, J=7.9 Hz, 1H), 7.49-7.46 (m, 2H), 7.41 (dd, J=8.1, 0.8 Hz, 1H), 7.12 (ddd, J=8.2, 7.1, 1.2 Hz, 1H), 7.03 (ddd, J=8.0, 7.1, 1.0 Hz, 1H), 6.93 (dd, J=2.2, 0.8 Hz, 1H). HRMS (EI) m/z calcd for C₁₄H₁₁ClN [M+H]⁺ 228.0580, found 228.0586.

### Embodiment 12

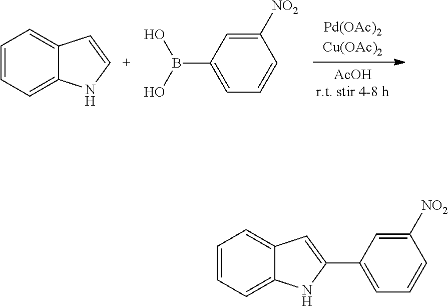 [SMILES: c1ccc2[nH]ccc2c1 ; O=[N+]([O-])c1cccc(B(O)O)c1 ; O=[N+]([O-])c1cccc(-c2cc3ccccc3[nH]2)c1]

Catalytic synthesis method: adding palladium acetate (0.5 equiv.) and copper acetate (0.5 equiv.) into 20 mL of glacial acetic acid solution of indole (1 equiv.) and 3-nitrophenylboronic acid (5 equiv.) in an air atmosphere, stirring at room temperature to react for 8 h, and detecting the reaction by TLC until the reaction is completed; conducting rotary evaporation for the reaction liquid for rotary evaporation of redundant glacial acetic acid and recovery; adding dichloromethane into the obtained black solid for dilution, and then extracting the mixture with (20 mL×3) saturated sodium bicarbonate solution; drying the organic extract with anhydrous magnesium sulfate, filtering, conducting rotary evaporation and concentration, separating by column chromatography, purifying and separating the product by taking silica gel of 200-300 meshes as a stationary phase and a mixed solvent of ethyl acetate and petroleum ether in different proportions as eluent, concentrating and drying to obtain yellowish powder. Physical properties and representation data of the obtained compound are as follows. The physical properties and the representation data of the obtained compound are as follows: yield 63%, m.p. 174.0-176.0° C.; ¹H NMR (400 MHz, DMSO) δ 11.86 (s, 1H), 8.71 (t, J=1.8 Hz, 1H), 8.32 (d, J=7.9 Hz, 1H), 8.14 (dd, J=8.1, 1.7 Hz, 1H), 7.74 (t, J=8.0 Hz, 1H), 7.58 (d, J=7.9 Hz, 1H), 7.44 (d, J=8.1 Hz, 1H), 7.19-7.12 (m, 2H), 7.04 (t, J=7.4 Hz, 1H). HRMS (EI) m/z calcd for C₁₄H₁₁N₂O₂ [M+H]⁺ 239.0830, found 239.0821.

### Embodiment 13

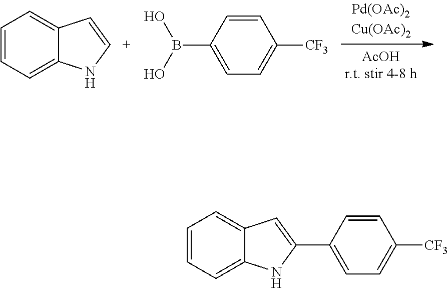 [SMILES: c1ccc2[nH]ccc2c1 ; OB(O)c1ccc(C(F)(F)F)cc1 ; FC(F)(F)c1ccc(-c2cc3ccccc3[nH]2)cc1]

Catalytic synthesis method: adding palladium acetate (0.5 equiv.) and copper acetate (0.5 equiv.) into 20 mL of glacial acetic acid solution of indole (1 equiv.) and 4-trifluoromethylphenylboric acid (1 equiv.) in an air atmosphere, stirring at room temperature to react for 8 h, and detecting the reaction by TLC until the reaction is completed; conducting rotary evaporation for the reaction liquid for rotary evaporation of redundant glacial acetic acid and recovery; adding dichloromethane into the obtained black solid for dilution, and then extracting the mixture with (20 mL×3) saturated sodium bicarbonate solution; drying the organic extract with anhydrous magnesium sulfate, filtering, conducting rotary evaporation and concentration, separating by column chromatography, purifying and separating the product by taking silica gel of 200-300 meshes as a stationary phase and a mixed solvent of ethyl acetate and petroleum ether in different proportions as eluent, concentrating and drying to obtain white powder. Physical properties and representation data of the obtained compound are as follows. The physical properties and the representation data of the obtained compound are as follows: yield 61%, m.p. 251.0-252.0° C.; ¹H NMR (400 MHz, DMSO) δ 11.73 (s, 1H), 8.06 (d, J=8.1 Hz, 2H), 7.80 (d, J=8.3 Hz, 2H), 7.57 (d, J=7.9 Hz, 1H), 7.44 (dd, J=8.1, 0.8 Hz, 1H), 7.15 (ddd, J=8.2, 7.1, 1.1 Hz, 1H), 7.09-6.98 (m, 2H). HRMS (EI) m/z calcd for C₁₅H₁₀F₃N [M−H]⁻260.0687, found 260.0682.

### Embodiment 14

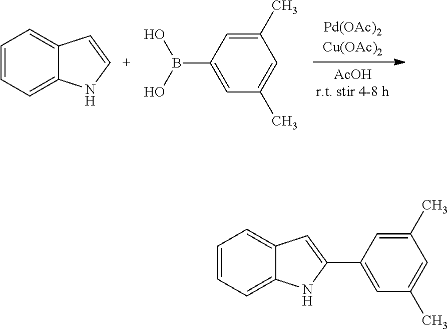 [SMILES: c1ccc2[nH]ccc2c1 ; Cc1cc(C)cc(B(O)O)c1 ; Cc1cc(C)cc(-c2cc3ccccc3[nH]2)c1]

Catalytic synthesis method: adding palladium acetate (0.5 equiv.) and copper acetate (0.5 equiv.) into 20 mL of glacial acetic acid solution of indole (1 equiv.) and 3,5-dimethylphenylboronic acid (1 equiv.) in an air atmosphere, stirring at room temperature to react for 8 h, and detecting the reaction by TLC until the reaction is completed; conducting rotary evaporation for the reaction liquid for rotary evaporation of redundant glacial acetic acid and recovery; adding dichloromethane into the obtained black solid for dilution, and then extracting the mixture with (20 mL×3) saturated sodium bicarbonate solution; drying the organic extract with anhydrous magnesium sulfate, filtering, conducting rotary evaporation and concentration, separating by column chromatography, purifying and separating the product by taking silica gel of 200-300 meshes as a stationary phase and a mixed solvent of ethyl acetate and petroleum ether in different proportions as eluent, concentrating and drying to obtain white powder. Physical properties and representation data of the obtained compound are as follows. The physical properties and the representation data of the obtained compound are as follows: yield 81%, m.p. 146.0-147.0° C.; ¹H NMR (400 MHz, DMSO) δ 11.46 (s, 1H), 7.55-7.46 (m, 3H), 7.39 (dd, J=8.1, 0.8 Hz, 1H), 7.08 (ddd, J=8.1, 7.1, 1.2 Hz, 1H), 6.98 (m, 2H), 6.84 (dd, J=2.1, 0.7 Hz, 1H), 2.34 (s, 6H). HRMS (EI) m/z calcd for C₁₆H₁₆N [M+H]⁺ 222.1283, found 222.1285.

### Embodiment 15

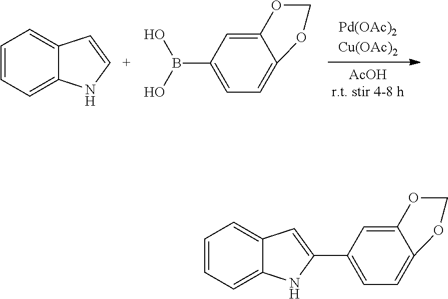 [SMILES: c1ccc2[nH]ccc2c1 ; OB(O)c1ccc2c(c1)OCO2 ; c1ccc2[nH]c(-c3ccc4c(c3)OCO4)cc2c1]

Catalytic synthesis method: adding palladium acetate (0.5 equiv.) and copper acetate (0.5 equiv.) into 20 mL of glacial acetic acid solution of indole (1 equiv.) and 3,4-(methylenedioxy)phenylboronic acid (5 equiv.) in an air atmosphere, stirring at room temperature to react for 8 h, and detecting the reaction by TLC until the reaction is completed; conducting rotary evaporation for the reaction liquid for rotary evaporation of redundant glacial acetic acid and recovery; adding dichloromethane into the obtained black solid for dilution, and then extracting the mixture with (20 mL×3) saturated sodium bicarbonate solution; drying the organic extract with anhydrous magnesium sulfate, filtering, conducting rotary evaporation and concentration, separating by column chromatography, purifying and separating the product by taking silica gel of 200-300 meshes as a stationary phase and a mixed solvent of ethyl acetate and petroleum ether in different proportions as eluent, concentrating and drying to obtain white powder. Physical properties and representation data of the obtained compound are as follows. The physical properties and the representation data of the obtained compound are as follows: yield 87%, m.p. 191.0° C.; ¹H NMR (400 MHz, DMSO) δ 11.37 (s, 1H), 7.48 (d, J=7.8 Hz, 1H), 7.44 (d, J=1.7 Hz, 1H), 7.36 (ddd, J=8.1, 2.6, 1.3 Hz, 2H), 7.08-7.04 (m, 1H), 7.01 (d, J=8.1 Hz, 1H), 6.98 (dd, J=10.9, 4.0 Hz, 1H), 6.79 (d, J=1.5 Hz, 1H), 6.06 (s, 2H). HRMS (EI) m/z calcd for C₁₅H₁₂NO₂ [M+H]⁺ 238.0868, found 238.0875.

### Embodiment 16

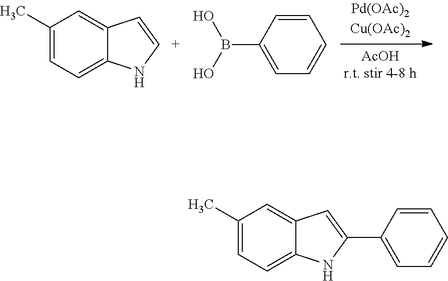

Catalytic synthesis method: adding palladium acetate (0.5 equiv.) and copper acetate (0.5 equiv.) into 20 mL of glacial acetic acid solution of 5-metlylindole (1 equiv.) and phenylboronic acid (1 equiv.) in an air atmosphere, stirring at room temperature to react for 8 h, and detecting the reaction by TLC until the reaction is completed; conducting rotary evaporation for the reaction liquid for rotary evaporation of redundant glacial acetic acid and recovery; adding dichloromethane into the obtained black solid for dilution, and then extracting the mixture with (20 mL×3) saturated sodium bicarbonate solution; drying the organic extract with anhydrous magnesium sulfate, filtering, conducting rotary evaporation and concentration, separating by column chromatography, purifying and separating the product by taking silica gel of 200-300 meshes as a stationary phase and a mixed solvent of ethyl acetate and petroleum ether in different proportions as eluent, concentrating and drying to obtain white powder. Physical properties and representation data of the obtained compound are as follows. The physical properties and the representation data of the obtained compound are as follows: yield 80%, m.p. 220.0-221.0° C.; ¹H NMR (400 MHz, DMSO) δ 11.37 (s, 1H), 7.83 (d, J=7.2 Hz, 2H), 7.44 (t, J=7.8 Hz, 2H), 7.29 (dd, J=8.1, 6.3 Hz, 3H), 6.92 (dd, J=8.2, 1.4 Hz, 1H), 6.80 (d, J=1.5 Hz, 1H), 2.36 (s, 3H). HRMS (EI) m/z calcd for C₁₅H₁₄N [M+H]⁺ 208.1126, found 208.1131.

### Embodiment 17

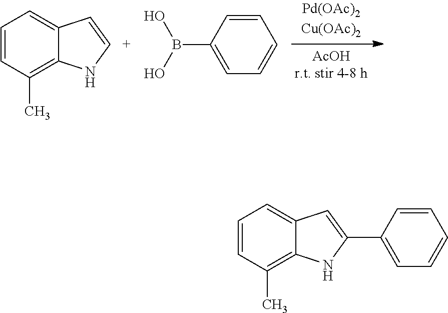

Catalytic synthesis method: adding palladium acetate (0.5 equiv.) and copper acetate (0.5 equiv.) into 20 mL of glacial acetic acid solution of 7-metlylindole (1 equiv.) and phenylboronic acid (1 equiv.) in an air atmosphere, stirring at room temperature to react for 8 h, and detecting the reaction by TLC until the reaction is completed; conducting rotary evaporation for the reaction liquid for rotary evaporation of redundant glacial acetic acid and recovery; adding dichloromethane into the obtained black solid for dilution, and then extracting the mixture with (20 mL×3) saturated sodium bicarbonate solution; drying the organic extract with anhydrous magnesium sulfate, filtering, conducting rotary evaporation and concentration, separating by column chromatography, purifying and separating the product by taking silica gel of 200-300 meshes as a stationary phase and a mixed solvent of ethyl acetate and petroleum ether in different proportions as eluent, concentrating and drying to obtain white powder. Physical properties and representation data of the obtained compound are as follows. The physical properties and the representation data of the obtained compound are as follows: yield 85%, m.p. 111.0-113.0° C.; ¹H NMR (400 MHz, DMSO) δ 11.03 (s, 1H), 7.89 (d, J=8.2 Hz, 2H), 7.38 (t, J=7.7 Hz, 2H), 7.31 (d, J=7.1 Hz, 1H), 7.24 (s, 1H), 6.90-6.78 (m, 3H), 2.50 (s, 3H). HRMS (EI) m/z calcd for C₁₅H₁₄N [M+H]⁺ 208.1126, found 208.1127.

### Embodiment 18

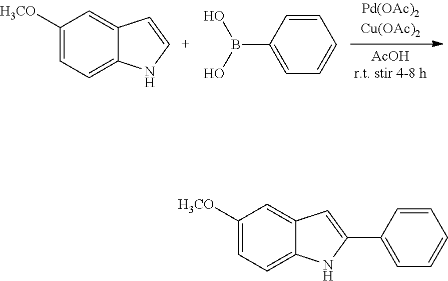 [SMILES: COc1ccc2[nH]ccc2c1 ; OB(O)c1ccccc1 ; COc1ccc2[nH]c(-c3ccccc3)cc2c1]

Catalytic synthesis method: adding palladium acetate (0.5 equiv.) and copper acetate (0.5 equiv.) into 20 mL of glacial acetic acid solution of 5-methoxyindole (1 equiv.) and phenylboronic acid (1 equiv.) in an air atmosphere, stirring at room temperature to react for 8 h, and detecting the reaction by TLC until the reaction is completed; conducting rotary evaporation for the reaction liquid for rotary evaporation of redundant glacial acetic acid and recovery; adding dichloromethane into the obtained black solid for dilution, and then extracting the mixture with (20 mL×3) saturated sodium bicarbonate solution; drying the organic extract with anhydrous magnesium sulfate, filtering, conducting rotary evaporation and concentration, separating by column chromatography, purifying and separating the product by taking silica gel of 200-300 meshes as a stationary phase and a mixed solvent of ethyl acetate and petroleum ether in different proportions as eluent, concentrating and drying to obtain white powder. Physical properties and representation data of the obtained compound are as follows. The physical properties and the representation data of the obtained compound are as follows: yield 86%, m.p. 164.0-165.0° C.; ¹H NMR (400 MHz, DMSO) δ 11.36 (s, 1H), 7.82 (d, J=7.5 Hz, 2H), 7.57-7.18 (m, 4H), 7.02 (s, 1H), 6.81 (s, 1H), 6.74 (d, J=8.3 Hz, 1H), 3.76 (s, 3H). HRMS (EI) m/z calcd for C₁₅H₁₄NO [M+H]⁺ 224.1075, found 223.1069.

### Embodiment 19

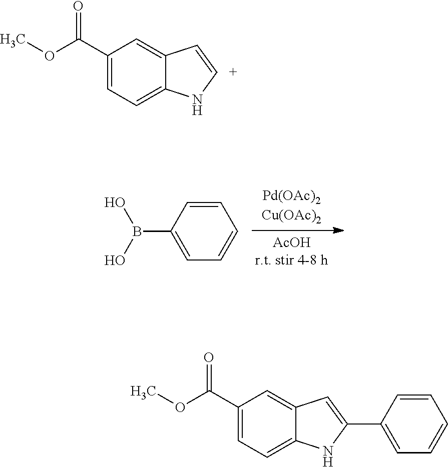 [SMILES: COC(=O)c1ccc2[nH]ccc2c1 ; OB(O)c1ccccc1 ; COC(=O)c1ccc2[nH]c(-c3ccccc3)cc2c1]

Catalytic synthesis method: adding palladium acetate (0.5 equiv.) and copper acetate (0.5 equiv.) into 20 mL of glacial acetic acid solution of 5-methyl formate indole (1 equiv.) and phenylboronic acid (3 equiv.) in an air atmosphere, stirring at room temperature to react for 8 h, and detecting the reaction by TLC until the reaction is completed; conducting rotary evaporation for the reaction liquid for rotary evaporation of redundant glacial acetic acid and recovery; adding dichloromethane into the obtained black solid for dilution, and then extracting the mixture with (20 mL×3) saturated sodium bicarbonate solution; drying the organic extract with anhydrous magnesium sulfate, filtering, conducting rotary evaporation and concentration, separating by column chromatography, purifying and separating the product by taking silica gel of 200-300 meshes as a stationary phase and a mixed solvent of ethyl acetate and petroleum ether in different proportions as eluent, concentrating and drying to obtain white powder. Physical properties and representation data of the obtained compound are as follows. The physical properties and the representation data of the obtained compound are as follows: yield 39%, m.p. 186.0-187.0° C.; ¹H NMR (400 MHz, DMSO)¹H NMR (400 MHz, DMSO) δ 11.94 (s, 1H), 8.25 (s, 1H), 7.88 (d, J=7.7 Hz, 2H), 7.75 (dd, J=8.5, 1.2 Hz, 1H), 7.53-7.45 (m, 3H), 7.35 (t, J=7.2 Hz, 1H), 7.06 (d, J=1.1 Hz, 1H), 3.85 (s, 3H). HRMS (EI) m/z calcd for C₁₅H₁₄NO [M+H]⁺ 252.1024, found 252.1029.

### Embodiment 20

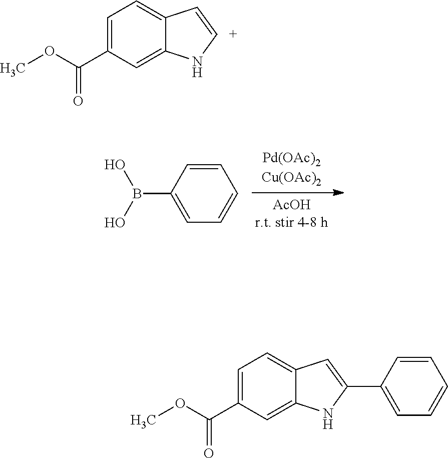 [SMILES: COC(=O)c1ccc2cc[nH]c2c1 ; OB(O)c1ccccc1 ; COC(=O)c1ccc2cc(-c3ccccc3)[nH]c2c1]

Catalytic synthesis method: adding palladium acetate (0.6 equiv.) and copper acetate (0.5 equiv.) into 20 mL of glacial acetic acid solution of 6-methyl formate indole (1 equiv.) and phenylboronic acid (3 equiv.) in an air atmosphere, stirring at room temperature to react for 8 h, and detecting the reaction by TLC until the reaction is completed; conducting rotary evaporation for the reaction liquid for rotary evaporation of redundant glacial acetic acid and recovery; adding dichloromethane into the obtained black solid for dilution, and then extracting the mixture with (20 mL×3) saturated sodium bicarbonate solution; drying the organic extract with anhydrous magnesium sulfate, filtering, conducting rotary evaporation and concentration, separating by column chromatography, purifying and separating the product by taking silica gel of 200-300 meshes as a stationary phase and a mixed solvent of ethyl acetate and petroleum ether in different proportions as eluent, concentrating and drying to obtain white powder. Physical properties and representation data of the obtained compound are as follows. The physical properties and the representation data of the obtained compound are as follows: yield 42%, m.p. 208.0-210.0° C.; ¹H NMR (400 MHz, DMSO) δ 11.95 (s, 1H), 8.07 (d, J=0.8 Hz, 1H), 7.98-7.76 (m, 2H), 7.63 (s, 2H), 7.51 (t, J=7.7 Hz, 2H), 7.39 (s, 1H), 7.03 (d, J=1.4 Hz, 1H), 3.87 (s, 3H). HRMS (EI) m/z calcd for C₁₆H₁₂NO₂ [M−H]⁻ 250.0868, found 250.0869.

### Embodiment 21

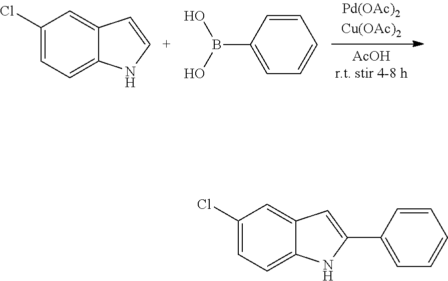 [SMILES: Clc1ccc2[nH]ccc2c1 ; OB(O)c1ccccc1 ; Clc1ccc2[nH]c(-c3ccccc3)cc2c1]

Catalytic synthesis method: adding palladium acetate (0.6 equiv.) and copper acetate (0.5 equiv.) into 20 mL of glacial acetic acid solution of 5-chloroindole (1 equiv.) and phenylboronic acid (1 equiv.) in an air atmosphere, stirring at room temperature to react for 8 h, and detecting the reaction by TLC until the reaction is completed; conducting rotary evaporation for the reaction liquid for rotary evaporation of redundant glacial acetic acid and recovery; adding dichloromethane into the obtained black solid for dilution, and then extracting the mixture with (20 mL×3) saturated sodium bicarbonate solution; drying the organic extract with anhydrous magnesium sulfate, filtering, conducting rotary evaporation and concentration, separating by column chromatography, purifying and separating the product by taking silica gel of 200-300 meshes as a stationary phase and a mixed solvent of ethyl acetate and petroleum ether in different proportions as eluent, concentrating and drying to obtain white powder. Physical properties and representation data of the obtained compound are as follows. The physical properties and the representation data of the obtained compound are as follows: yield 69%, m.p. 199.0-200.0° C.; ¹H NMR (400 MHz, DMSO) δ 11.74 (s, 1H), 7.95-7.79 (m, 2H), 7.57 (d, J=2.0 Hz, 1H), 7.47 (t, J=7.7 Hz, 2H), 7.40 (d, J=8.6 Hz, 1H), 7.35 (d, J=7.4 Hz, 1H), 7.09 (dd, J=8.6, 2.1 Hz, 1H), 6.89 (d, J=1.6 Hz, 1H). HRMS (EI) m/z calcd for C₁₄H₉ClN [M−H]⁻224.0424, found 224.0434.

### Embodiment 22

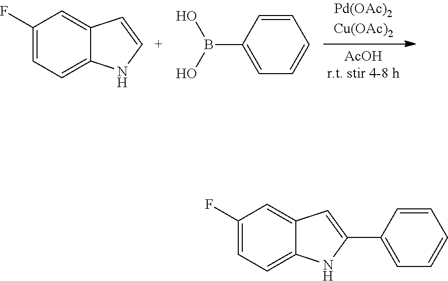 [SMILES: Fc1ccc2[nH]ccc2c1 ; OB(O)c1ccccc1 ; Fc1ccc2[nH]c(-c3ccccc3)cc2c1]

Catalytic synthesis method: adding palladium acetate 0.6 equiv.) and copper acetate (0.5 equiv.) into 20 mL of glacial acetic acid solution of 5-fluoroindole (1 equiv.) and phenylboronic acid (1 equiv.) in an air atmosphere, stirring at room temperature to react for 8 h, and detecting the reaction by TLC until the reaction is completed; conducting rotary evaporation for the reaction liquid for rotary evaporation of redundant glacial acetic acid and recovery; adding dichloromethane into the obtained black solid for dilution, and then extracting the mixture with (20 mL×3) saturated sodium bicarbonate solution; drying the organic extract with anhydrous magnesium sulfate, filtering, conducting rotary evaporation and concentration, separating by column chromatography, purifying and separating the product by taking silica gel of 200-300 meshes as a stationary phase and a mixed solvent of ethyl acetate and petroleum ether in different proportions as eluent, concentrating and drying to obtain white powder. Physical properties and representation data of the obtained compound are as follows. The physical properties and the representation data of the obtained compound are as follows: yield 56%, m.p. 178.0-179.0° C.; ¹H NMR (400 MHz, CDCl₃) δ 8.29 (s, 1H), 7.66-7.60 (m, 2H), 7.47-7.40 (m, 2H), 7.36-7.31 (m, 1H), 7.27 (m, 2H), 6.97-6.88 (m, 1H), 6.77 (s, 1H). HRMS (EI) m/z calcd for C₁₄H₉FN [M−H]⁻ 210.0719, found 210.0721.

### Embodiment 23

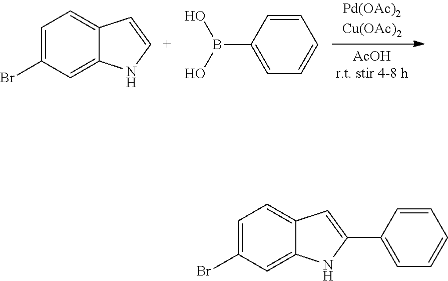 [SMILES: Brc1ccc2cc[nH]c2c1 ; OB(O)c1ccccc1 ; Brc1ccc2cc(-c3ccccc3)[nH]c2c1]

Catalytic synthesis method: adding palladium acetate (0.5 equiv.) and copper acetate (0.6 equiv.) into 20 mL of glacial acetic acid solution of 6-bromoindole (1 equiv.) and phenylboronic acid (1 equiv.) in an air atmosphere, stirring at room temperature to react for 8 h, and detecting the reaction by TLC until the reaction is completed; conducting rotary evaporation for the reaction liquid for rotary evaporation of redundant glacial acetic acid and recovery; adding dichloromethane into the obtained black solid for dilution, and then extracting the mixture with (20 mL×3) saturated sodium bicarbonate solution; drying the organic extract with anhydrous magnesium sulfate, filtering, conducting rotary evaporation and concentration, separating by column chromatography, purifying and separating the product by taking silica gel of 200-300 meshes as a stationary phase and a mixed solvent of ethyl acetate and petroleum ether in different proportions as eluent, concentrating and drying to obtain white powder. Physical properties and representation data of the obtained compound are as follows. The physical properties and the representation data of the obtained compound are as follows: yield 67%, m.p. 187.0° C.; ¹H NMR (400 MHz, DMSO) δ 11.70 (s, 1H), 7.85 (dd, J=8.3, 1.0 Hz, 2H), 7.55-7.53 (m, 1H), 7.51-7.45 (m, 3H), 7.34 (t, J=7.4 Hz, 1H), 7.13 (dd, J=8.4, 1.8 Hz, 1H), 6.93 (d, J=1.5 Hz, 1H). HRMS (EI) m/z calcd for C₁₄H₉BrN [M−H]⁻ 269.9918, found 269.9935.

### Test Example

### I. Detection of Minimum Inhibitory Concentration (MIC) of Compounds and Azole Drugs by Microdilution Method

The combination of the compound of the present invention and the azole drugs, a synergistic effect on inhibiting the proliferation of clinical multi-drug resistant Candida albicans CA10 and the inhibition of standard sensitive strains are detected by checkerboard microdilution assay.

### 1.1 Experiment Material

Used bacteria: including drug-resistant bacteria and sensitive bacteria: the used sensitive strains include Cryptococcus neoformans BNCC225501, Candida albicans BNCC186382, Aspergillus fumigatus Fresenius BNCC338385, Escherichia coli BNCC336902, Staphylococcus aureus BNCC186335 and Pseudomonas aeruginosa Migula BNCC337940, and the used drug-resistant Candida albicans is clinically isolated azole multi-drug resistant Candida albicans (CA10) donated by Qianfoshan Hospital in Shandong Province. The strains are stored in normal saline containing 20% glycerol at −80° C. for a long time. Before operation, the strains are lined and transferred to a YPD solid plate, and subjected to static culture at 30° C. for 24 h; single colonies are selected and transferred to a YPD liquid medium, and cultured at 200 rpm at 30° C. overnight; and the overnight cultured bacterial solution is inoculated into a new YPD liquid medium at a ratio of about 1:100, and subjected to shaking culture at 200 rpm at 30° C. for 4 h so that Candida albicans is in a logarithmic phase.

Culture medium: the culture medium used by the microdilution method is RPMI-1640 culture medium (Gibco).

Drugs: 3-(N-morpholino)propanesulfonic acid (MOPS), dimethyl sulfoxide (DMSO) and fluconazole.

1.2 Operation and results of detection of minimum inhibitory concentration (MIC) of the compounds and the azole drugs by the microdilution method: the MIC of the synthesized compound and the azole drug fluconazole for the standard strains is detected by the microdilution method according to fungal sensitivity detection method (M27-A3) established by the Clinical and Laboratory Standards Institute (CLSI). Specific steps are as follows: the prepared strain concentration is adjusted to 0.5-2.5×10³ CFU/mL by using RPMI-1640 medium. 200 μL of bacterial solution containing the highest concentration of the drug to be detected is added into the first well of a 96-well plate, and 100 μL of blank bacterial solution without drug is added into each of the other wells; the bacterial solution is diluted to a series of concentration gradients by a double gradient dilution method; the well plate is subjected to static culture at constant temperature of 35° C. for 24 h; after the absorbance (OD) of each concentration gradient at 570 nm is detected by a microplate reader and the absorbance of a blank medium is subtracted, the growth rate=dosing hole value/control hole value ×100%, the inhibition rate=1−growth rate, and the minimum concentration with the inhibition rate greater than 80% is the minimum inhibitory concentration (MIC) value.

TABLE 1
MIC Results of Indole Derivatives against Sensitive Bacteria
MIC (μg/ml)

| | Fungi | | | Bacteria | | |
|---|---|---|---|---|---|---|
| | | | Aspergillus | | | Pseudomonas |
| | Candida | Cryptococcus | fumigatus | Staphylococcus | Escherichia | aeruginosa |
| Compound | albicans | neoformans | Fresenius | aureus | coli | Migula |
| Fluconazole | 2 | 2 | 2 | | | |
| Amphotericin | 2 | 64 | 16 | | | |
| B | | | | | | |
| Streptomycin | | | | 2 | 2 | 2 |
| 1 | 16 | 4 | 8 | 64 | 4 | 8 |
| 2 | 4 | 2 | 16 | 8 | 8 | 256 |
| 3 | 256 | 256 | 256 | 256 | 256 | 64 |
| 4 | 16 | 16 | 8 | 8 | 16 | 16 |
| 5 | 16 | 256 | 16 | 32 | 16 | 256 |
| 6 | >256 | 256 | >256 | >256 | >256 | >256 |
| 7 | 256 | 256 | 256 | 8 | 8 | 256 |
| 8 | 256 | 256 | 256 | 256 | 8 | 256 |
| 10 | >256 | >256 | >256 | >256 | >256 | >256 |
| 11 | >256 | >256 | >256 | >256 | >256 | >256 |
| 12 | 4 | 8 | 4 | 256 | 8 | 2 |
| 13 | 256 | 256 | 256 | 256 | 256 | 16 |
| 15 | 256 | 256 | 256 | 256 | 256 | 16 |
| 16 | 256 | 256 | 256 | 256 | 16 | 256 |
| 18 | 256 | 256 | 256 | 256 | 256 | 256 |
| 19 | 8 | 256 | 16 | 8 | 256 | 32 |
| 20 | 32 | 256 | 256 | 32 | 256 | 8 |
| 21 | >256 | >256 | >256 | >256 | >256 | >256 |
| 22 | 256 | 8 | 256 | 16 | 32 | 256 |
| 23 | 256 | 256 | 256 | 256 | 256 | 8 |

TABLE 2
MIC Results of Indole Derivatives against Drug Resistant Bacteria
MIC₈₀

| Compound | Drug-resistant Candida albicans CA10 |
|---|---|
| Fluconazole | >128 |
| 1 | >256 |
| 2 | 32 |
| 3 | >256 |
| 4 | 32 |
| 5 | 256 |
| 6 | >256 |
| 7 | >256 |
| 8 | >256 |
| 10 | >256 |
| 11 | >256 |
| 12 | >256 |
| 13 | >256 |
| 15 | >256 |
| 16 | >256 |
| 18 | >256 |
| 19 | >256 |
| 20 | >256 |
| 21 | >256 |
| 22 | >256 |
| 23 | >256 |

### II. Detection of the Antifungal Effect of the Combination of the Compound and Azoles by Checkerboard Microdilution Assay:

### 2.1 Experimental Method

The concentration of Candida albicans CA10 is adjusted to 0.5-2.5×103 CFU/mL by using RPMI-1640 medium, and the diluted bacterial solution is used for preparing a working solution of the compound and the azole drug with double concentration gradient. 50 μl of working solution of double concentration of azole drugs and working solution of double concentration of the compound to be detected at each concentration gradient are added in the longitudinal (A-H) direction and transverse (1-12) direction of the 96-well plate respectively, so that the final drug concentration range of fluconazole is 4-64 μg/ml, and the drug concentration range of the compound is 256-16 μg/ml. The 96-well plate is cultured in a constant temperature incubator at 35° C. for 24 h. The amount of viable bacteria is detected with an XTT cell proliferation assay kit, and the absorbance of each well at 490 nm wavelength is detected by the microplate reader. Growth rate=dosing well value/control well value×100%, and inhibition rate=1−growth rate. The MIC when the inhibition rate is about 80% is a break point of the combination of two drugs.

### 2.2 FICI Evaluation Model:

The combination data of the compound and the azole drug collected by the checkerboard microdilution assay are analyzed by an FICI (fractional inhibitory concentration index) model, and the interaction effects are evaluated.

The FICI model can be expressed by the following formula:

$$FICI = FICA + FICB = MICAB / MICA + MICBA / MICB$$

Wherein: MICA and MICB are MIC values of drug A and drug B used separately, while MICAB and MICBA are the concentrations of drug A and drug B corresponding to the minimum effective dose of the combination of drug A and drug B.

### Determination Criteria:

if FICI≤0.5, the interaction between drug A and drug B is a synergistic effect; FICI>4, antagonistic effect; 0.5<FICI≤4, no correlation effect.

When the sensitivity of the drug to Candida albicans is determined by a microliquid dilution method, if MIC of fluconazole is <8 μg/ml, it is a sensitive bacterium, if ≥64 μg/ml, it is a drug-resistant bacterium, and if 16-32 μg/ml, it is a dose-dependent sensitive bacterium.

### 2.3 Experimental Result

The results shows that: after evaluation by the FICI model, it is found that compounds 2 and 5 show the synergistic effect with the azole drug fluconazole, and the calculated result of compound 4 shows no correlation, but can improve the sensitivity of clinical multi-drug resistant strain CA10 to the azole drug, so that the combination concentration is reduced to be less than the drug-resistant concentration, and the effect of reversing the drug resistance can be achieved (Table 3).

TABLE 3
Synergistic Effect of Combination of Compound and Azole
Drug against Drug-Resistant Fungus Candida Albicans

| | MICs of drugs (μg/ml) | | | | | |
|---|---|---|---|---|---|---|
| | Used separately | | Combined | | FICI model | |
| Compound | Azole | Compound | Azole | Compound | FICI | Combination effect |

Fluconazole

| | | | | | | |
|---|---|---|---|---|---|---|
| 2 | >128 | 32 | 8 | 8 | 0.313 | Synergistic |
| 4 | >128 | 32 | 8 | 16 | 0.563 | No correlation |
| 5 | >128 | >256 | 8 | 64 | 0.313 | Synergistic |

### III. Cytotoxicity Test of Compounds 2, 4 and 5 for RAW 264.7

### 3.1 Experiment Material

3.1.1 Cell line: RAW 264.7 murine macrophage cells are donated by Shihezi University College of Pharmacy, and cultured in a 37° C. constant temperature incubator with high glucose DMEM medium (Gibco), 10% FBS serum (Gibco) and 5% CO2, and subculturing and related experimental operation are conducted when cell fusion degree reaches about 70-80%.

3.1.2 Materials and reagents: fetal bovine serum (American BI company), DMEM high glucose medium (American Gibco company), penicillin-streptomycin solution (Shanghai Solarbio company), PBS buffer solution (American Gibco company), isopropyl alcohol (analytically pure), dimethyl sulfoxide (DMSO) (Shanghai Solarbio company), and MTT powder (Shanghai Solarbio company).

### 3.2 Experimental Method

### 3.2.1 MTT Experiment

Logarithmic RAW 264.7 cells are taken; cell suspension is prepared with a complete medium; the concentration of the cell suspension is adjusted to about 5×104 cells/mL; 200 μl of cell suspension is added into each hole of the 96-well culture plate and cultured in 5% CO2 in an incubator at 37° C. for about 24 h until the cells grow to the cell wall (at density of 50%); the medium is absorbed by a 5 ml needle tube or negative pressure; RAW 264.7 is divided into a blank group, a solvent control group and a drug administration group; in the blank group, there is no cell, and a medium, MTT and DMSO are added; in the solvent control group, cells, the same concentration of drug dissolving media, culture solution, MTT and DMSO are added; in the drug administration group, medical solution for diluting the concentration gradient with serum-free medium (containing 1% PE) is added; each concentration shall set at least 3 complex wells; 100 μl of medical solution is added into each well; and the culture plate is continued to be incubated in the incubator for 24 h. After 10 μl of MTT solution is added into each well along the wall of the well away from light (to ensure the uniformity of MTT addition, MTT can be diluted in an incomplete medium and then added into the 96-well plate in proportion to ensure that the added MTT is 10% of the total volume) and incubated in the incubator for 2-4 h, the supernatant is carefully absorbed. 100 μl of DMSO is added into each well to dissolve formazan crystals, and shaken in a shaker for 10 min at low speed to fully dissolve the crystals. The absorbance (OD) value of each well at 490 nm is determined by the microplate reader, and the cell viability is calculated by the formula: Cell viability (%)=(OD drug administration group−OD blank group)/(OD solvent control group−OD blank group)× 100%.

### 3.3 Experimental Result

OD values at 490 nm are determined by the experimental method in each group, and the data are subjected to one-way analysis of variance by Graphpad prism 8.0 software.

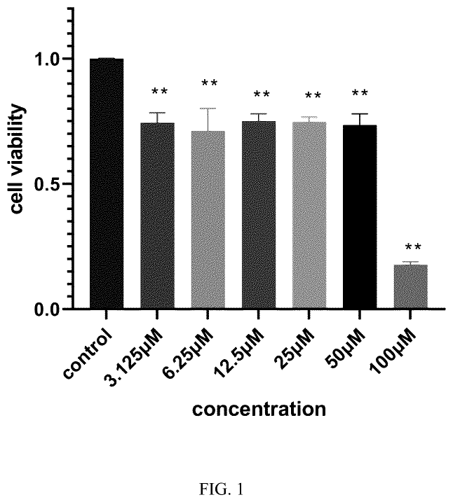

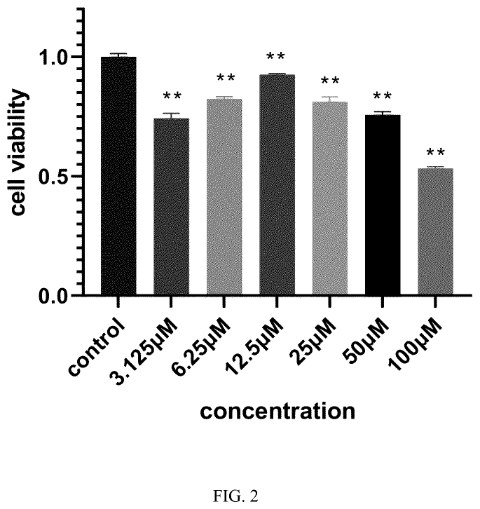

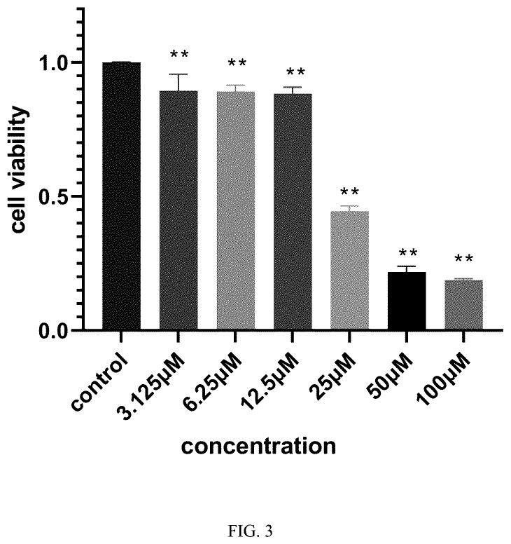

To explore the influences of 2-phenylindole derivatives on survival of mouse macrophages RAW 264.7, results of processing RAW 264.7 cells with different concentrations of 2-phenylindole derivatives for 24 h are shown in FIG. 1, FIG. 2 and FIG. 3. FIG. 1 shows the influence of compound 2 on RAW 264.7 cell viability, FIG. 2 shows the influence of compound 4 on RAW 264.7 cell viability and FIG. 3 shows the influence of compound 5 on RAW 264.7 cell viability (compared with the control group, * and ** indicate P<0.05 and P<0.01 in the figures respectively). The table shows that compound 5 may inhibit cell proliferation in a concentration-dependent way, and compounds 2, 4, and 5 have no obvious toxicity to RAW 264.7 cells (P<0.05, Table 4).

TABLE 4
Influence of Three Compounds on RAW 264.7 Cell Viability

| Compound | Structural formula | IC₅₀ (uM) |
|---|---|---|
| 2 | 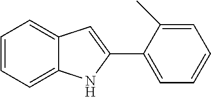 [SMILES: Cc1ccccc1-c1cc2ccccc2[nH]1] | 50 |
| 4 | 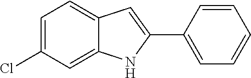 [SMILES: Clc1ccc2cc(-c3ccccc3)[nH]c2c1] | 100 |
| 5 | 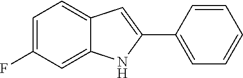 [SMILES: Fc1ccc2cc(-c3ccccc3)[nH]c2c1] | 12.5 |

Each embodiment in the description is described in a progressive way. The difference of each embodiment from each other is the focus of explanation. The same and similar parts among all of the embodiments can be referred to each other. For the device disclosed by the embodiment, because the device corresponds to the method disclosed by the embodiment, the device is simply described. Refer to the description of the method part for the related part.

The above description of the disclosed embodiments enables those skilled in the art to realize or use the present invention. Many modifications to these embodiments will be apparent to those skilled in the art. The general principle defined herein can be realized in other embodiments without departing from the spirit or scope of the present invention. Therefore, the present invention will not be limited to these embodiments shown herein, but will conform to the widest scope consistent with the principle and novel features disclosed herein.

What is claimed is:

1. A preparation method of 2-phenylindole derivatives, comprising: adding palladium acetate and copper acetate into a glacial acetic acid solution of formula I and formula II, stirring at room temperature, and purifying to obtain a target product (formula III): 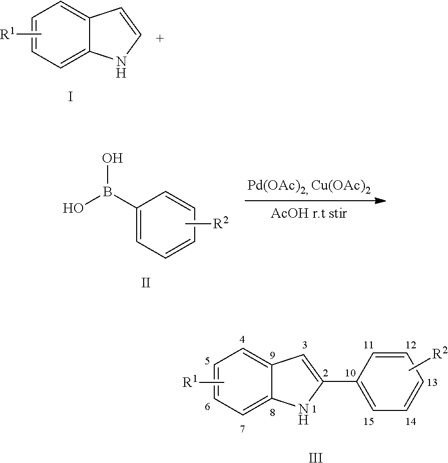
wherein
R¹ is substituted at 5, 6 or 7 site, and substituents are selected from F, Cl, OCH₃, CH₃, COOCH₃ and N—CH₃;
R² is substituted at 11, 12, 13 or 14 site, and substituents are selected from F, Cl, CH₃, OCH₃, CF₃, NO₂ or CH₂O₂.

2. The preparation method of 2-phenylindole derivatives according to claim 1, wherein a molar ratio of the formula I: formula II in the glacial acetic acid solution of the formula I and the formula II is 1: 1-10.

3. The preparation method of 2-phenylindole derivatives according to claim 1, wherein a molar ratio of the formula I: palladium acetate is 1: 0.1-0.8; and a molar ratio of the formula I: copper acetate is 1: 0.1-0.8.

4. The preparation method of 2-phenylindole derivatives according to claim 1, wherein stirring time is 6-10 h.

5. The preparation method of 2-phenylindole derivatives according to claim 1, wherein the purification specifically comprises: conducting rotary evaporation for the reaction liquid after the end of the reaction for rotary evaporation of redundant glacial acetic acid and recovering, adding dichloromethane into obtained black solid for diluting, then extracting the mixture with saturated sodium bicarbonate solution, drying the organic extract with anhydrous magnesium sulfate, filtering, conducting rotary evaporation and concentration, and finally separating and purifying by column chromatography silica gel to obtain a target product.

6. The preparation method of 2-phenylindole derivatives according to claim 5, wherein the separation of the column chromatography silica gel uses silica gel of 300-400 meshes as a stationary phase, and a mixed solvent of ethyl acetate and petroleum ether in different proportions is used as eluent.

7. An application of the 2-phenylindole derivative prepared by the preparation method of claim 1 in preparation of antifungal reagents or antibacterial reagents.

8. An application of the 2-phenylindole derivative prepared by the preparation method of claim 2 in preparation of antifungal reagents or antibacterial reagents.

9. An application of the 2-phenylindole derivative prepared by the preparation method of claim 3 in preparation of antifungal reagents or antibacterial reagents.

10. An application of the 2-phenylindole derivative prepared by the preparation method of claim 4 in preparation of antifungal reagents or antibacterial reagents.

11. An application of the 2-phenylindole derivative prepared by the preparation method of claim 5 in preparation of antifungal reagents or antibacterial reagents.

12. An application of the 2-phenylindole derivative prepared by the preparation method of claim 6 in preparation of antifungal reagents or antibacterial reagents.

13. The application of the 2-phenylindole derivative in preparation of antifungal reagents or antibacterial reagents according to claim 7, wherein fungi comprise Candida albicans, Cryptococcosis and Aspergillus fumigatus Fresenius; and bacteria comprise Staphylococcus aureus, Escherichia coli or Pseudomonas aeruginosa Migula.

14. The application of the 2-phenylindole derivative in preparation of antifungal reagents or antibacterial reagents according to claim 7, wherein the fungi are drug-resistant bacteria or sensitive bacteria; the antifungal reagents also comprise azole antifungal drugs; the azole antifungal drugs comprise ketoconazole, itraconazole or fluconazole; and a mass ratio of the azole antifungal drugs to the 2-phenylindole derivative is 1-16: 64-4.
